(19) United States
(12) Patent Application Publication
(10) Pub. No.: US 2013/0281882 A1
(43) Pub. Date: Oct. 24, 2013

(54) LANCET DEVICE

(71) Applicants: Geun Sig Cha, Seoul (KR); Hakhyun Nam, Seoul (KR); Eun-Jong Cha, Cheongju-si (KR); Kyung-Ah Kim, Cheongju-si (KR)

(72) Inventors: Geun Sig Cha, Seoul (KR); Hakhyun Nam, Seoul (KR); Eun-Jong Cha, Cheongju-si (KR); Kyung-Ah Kim, Cheongju-si (KR)

(73) Assignee: I-SENS, INC., Seoul (KR)

(21) Appl. No.: 13/995,152

(22) PCT Filed: Sep. 20, 2011
(86) PCT No.: PCT/KR2011/006936

§ 371 Date: Jun. 17, 2013

(30) Foreign Application Priority Data
Dec. 15, 2010 (KR) 10-2010-0128560

(51) Int. Cl. A61B 5/15; A61B 5/151

(52) U.S. Cl. CPC A61B 5/150664; A61B 5/15188
USPC 600/583

(57) ABSTRACT

The present invention relates to a lancet device that can solve the problem of falling or losing a cover when mounting or separating a lancet and reduce the pain that accompanies blood-taking. A lancet device according to an exemplary embodiment of the present invention includes: a lancet holder having a lancet seat to mount a lancet at one end; an operating mechanism disposed at the other end of the lancet holder and loading and releasing the lancet holder along a predetermined path; and an a housing receiving the operating mechanism and having one end coupled to the lancet holder, and further includes a cover that covers the housing so that the lancet holder is received in the housing, is at least partially connected with the housing in order not to separate from the housing in use, and has protrusions on the front part.

### FIELD OF THE INVENTION

The present invention relates to a lancet device. More particularly, the present invention relates to a lancet device that can solve the problem of falling or losing a cover when mounting or separating a lancet and reduce the pain that accompanies blood-taking.

### DESCRIPTION OF THE RELATED ART

In general, chronic diabetics have to measure the blood glucose level by performing a blood sugar test by themselves everyday at home and to perform disease control in order to keep a predetermined blood glucose level.

They have to collect blood for the blood glucose test, and this case, generally, they stick a disposable lancet into the skin of a portion, usually a finger, of their bodies, take and put a small amount of capillary blood onto a strip, and then measure the blood glucose level using a blood glucose meter with the strip mounted.

A lancing device is generally used as the device for taking blood.

The lancing device is composed of a lancet holder mounted with a disposable lancet, a cover that covers a lancet and has a hole through which only the tip of a needle protrudes to penetrate a skin, and a spring and a releasing member that provide a penetration force. The disposable lancet has a needle at one end of a lancet body and a protection cap is combined with the needle.

According to the lancing devices having this configuration in the related art, a user removes the cover from a lancing device, mounts a disposable lancet onto the lancet holder, attaches the cover with the spring compressed, brings the lancet in close contact with a portion with many capillaries such as fingers, and then releases the disposable lancet by pulling a releasing switch, such that the lancet penetrates the skin.

However, it is troublesome to use the lancing devices, because it is required to separate a cover from the lancing devices and place it at a side or hold it for a moment, when mounting or separating a lancet, and the cover is small, such that the cover is likely to be lost when a user falls it by mistake.

Further, since the needle penetrates a skin, a pain accompanies, but it is taken as a matter of course to be accepted in order to take blood.

### DISCLOSURE

### Technical Problem

The present invention has been made in an effort to provide a lancet device having advantages of being able to solve the problem of falling or losing a cover when mounting or separating a lancet and reduce the pain that accompanies blood-taking.

### Technical Solution

An exemplary embodiment of the present invention provides a lancet device, which includes: a lancet holder having a lancet seat to mount a lancet at one end; an operating mechanism disposed at the other end of the lancet holder and loading and releasing the lancet holder along a predetermined path; and an a housing receiving the operating mechanism and having one end coupled to the lancet holder, and further includes a cover that covers the housing so that the lancet holder is received in the housing, is at least partially connected with the housing in order not to separate from the housing in use, and has protrusions on the front part.

According to the present invention described above, the following effects are achieved.

First, it is possible to remove the problem in that a user has to separate the cover from the lancet device and then places it or hold it in a hand or the problem of losing the cover while mounting and separating the lancet.

Second, a user can sensuously feel the position to take blood from, by means of the protrusions, and easily takes blood because the blood vessels around the portion to take blood from are pressed and blood is collected, and the pain reduces when the lancet is released.

Third, since the protrusions are alternately arranged along a plurality of coaxial circles and the contact area is wide, it is possible to stimulate many nerves around the portion to take blood from at a time, such that it is possible to press more blood vessels around the portion and is easy to take more blood.

### DESCRIPTION OF DRAWINGS

FIG. 1 is a perspective view of a lancet device according to an exemplary embodiment of the present invention.

FIG. 2 is an exploded perspective view of the lancet device according to an exemplary embodiment of the present invention.

FIG. 3 is a cross-sectional view of the assembly of the parts shown in FIG. 1.

FIG. 4 is a partial enlarged view of FIG. 1.

FIG. 5 is a status view for illustrating how to use a cover of the lancet device according to an exemplary embodiment of the present invention.

### BEST MODE

Hereinafter, exemplary embodiments of the present invention will be described in detail with reference to the accompanying drawings. First, when giving the components in the drawings reference numerals, it should be noted that the same reference numerals are given to reference numerals as same as possible even if they are shown in different drawings. Further, the in description of the present invention, the detailed description of related well-known configurations and functions is not provided, when it is determined as making the scope of the present invention unclear.

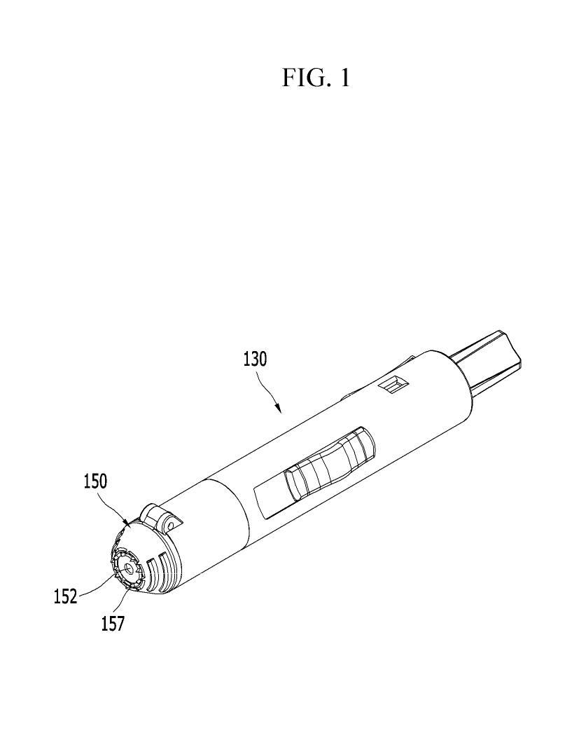

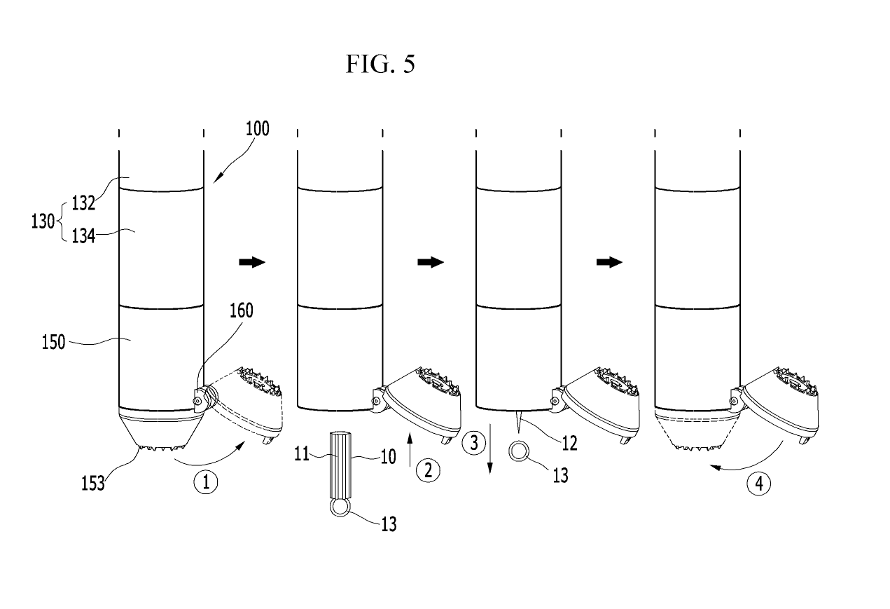

Referring to FIGS. 1 and 5, a lancet device 100 according to an exemplary embodiment of the present invention includes a lancet holder 110, an operating mechanism 120, a housing 130, an operation switch 170, a cover 150, a coupling portion 160, and a depth adjusting member 140.

The lancet holder 110 has a lancet seat 111 to mount a lancet 10 at one end and is disposed at a side in the housing 130.

The lancet 10, which is disposable, has a lancet needle 12 at one end of a lancet body 11 with a predetermined length and the lancet needle 12 is combined with a protection cap 13 (see FIG. 5).

The protection cap 13 is coupled to one end of the lancet needle 12 by insert injection, and according to this configuration, the protection cap 13 can be separated from the lancet needle 12 even by a small force.

The operating mechanism 120 is disposed at the other end of the lancet holder 110 and has a structure for loading and releasing the lancet holder 110 along a predetermined path, and for this operation, it includes a plurality of springs 121, 122, and 123, as shown in FIG. 1.

The housing 130, a cylinder sized to be portable by a user, receives the operating mechanism 120, with the lancet holder 110 coupled to one end, and the operation switch 170 operating with the operating mechanism 120 is disposed at a predetermined position.

The operation switch 170 is connected to the operating mechanism 120 and operates the operating mechanism 120 by being pressed.

When a user presses the operation switch 170, the springs 121, 122, and 123 are instantaneously compressed, and extend and hit a movable shaft 125 under which a shock transmission rod 124 is positioned, the movable shaft 125 hits the lancet holder 110 holding the lancet 10, and the lancet 10 protrudes outward.

The cover 150 covers an end of the housing 130, has a through-hole 152 at the center through which the lancet needle 12 of the lancet 10 instantaneously protrudes, and has a simple opening/closing structure such as a hinged door, different from the way of mounting a lancet after separating a cover in the existing products.

The coupling portion 160 hinges one end of the housing 130 with the cover 150, such that the cover 150 pivots to open/close at 180 degrees, as shown in FIG. 5.

According to this structure, it is possible to remove the problem in that a user has to separate the cover 150 from the lancet device 100 and then places it or hold it in a hand or the problem of losing the cover 150 while mounting and separating the lancet 10.

To this end, the cover has a front part 151 and a locking protrusion 155.

The through-hole 152 is formed at the center of the front part 151 so that the lancet needle 12 can instantaneously protrude out of the cover 150 and take blood by means of the operating mechanism 120.

The locking protrusion 155 is formed at the other end of the front part 151 in the shape corresponding to a fitting groove 131 formed at one end of the housing 130.

Therefore, it is possible to keep the cover 150 closed by fitting the locking protrusion 155 into the fitting groove 131 without adding a complicated configuration.

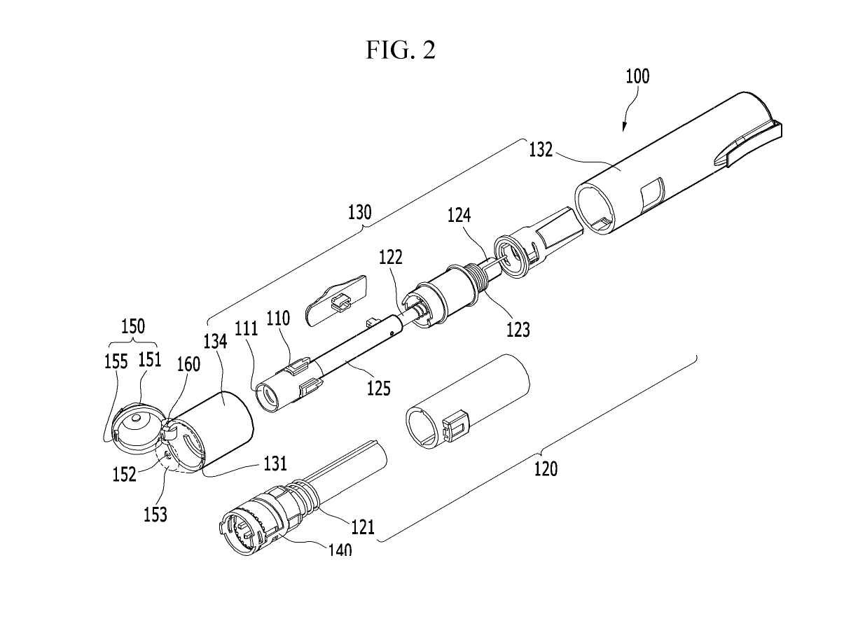

Further, as shown in FIGS. 1 and 2, a plurality of protrusions 153 is radially formed at regular angles in the shape of a gear on the front part 151 of the cover 150.

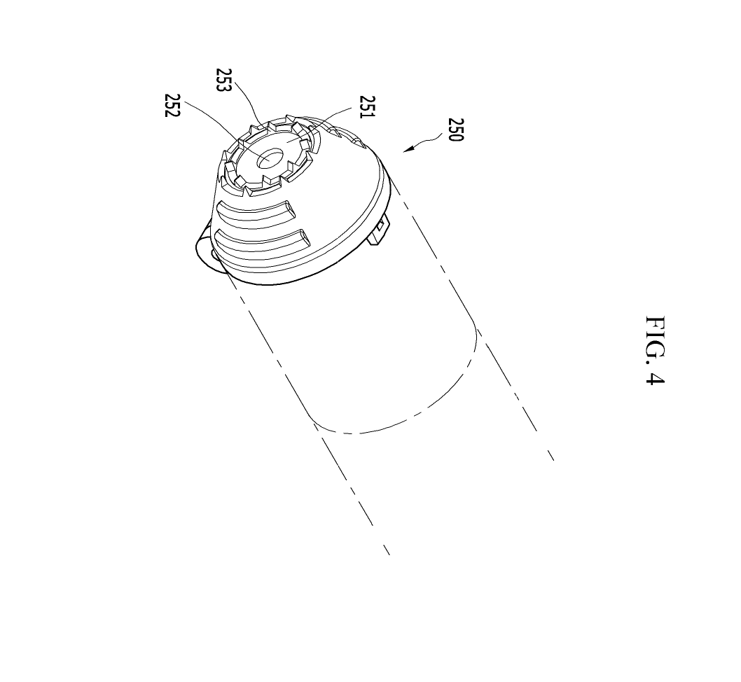

In particular, as shown in FIG. 4, it is preferable that the protrusions 153 have a sharp triangular shape and are arranged along a plurality of coaxial circles formed along the edge of the through-hole 152 of the front part 151 of the cover 150, that is, alternately in two lines to increase the area that comes in contact with a skin.

The protrusions 150 press the skin and stimulate the nerves of the portion to take blood from, before the lancet needle 12 penetrates the skin in taking blood.

Therefore, a user can sensuously feel the position to take blood from, and easily takes blood because the blood vessels around the portion to take blood from are pressed and blood is collected, and the pain reduces when the lancet 10 is released.

The depth adjusting member 140 adjusts the penetration depth under the skin of the lancet needle 12.

To this end, as shown in FIG. 1, the housing 130 includes a first housing part 132 receiving the operating mechanism 120 and a second housing part 134 rotating about the axis of the first housing part 132 and combined with the depth adjusting member 140 therein.

The second housing part 132 is combined with the cover 150 and the depth adjusting member 140 and the distance between the through-hole 152 and the lancet needle 12 is adjusted by rotating the cover 150 or the second housing part 132.

How to use the cover 150 of the lancet device 100 of the present invention is described hereafter with reference to FIG. 5.

First, a user opens the cover 150 from the lancet device 100 by holding and turning the cover 150 by 180 degrees to the coupling portion 160 (\textcircled{1}) and then mounts the lancet 10 onto the lancet seat 111 (see FIG. 2) of the lancet holder 110 (\textcircled{2}),

Then, the user separates the protection cap 13 from the lancet 10 to expose the lancet needle 12 to the outside (\textcircled{3}), and then mounts the cover 150 to one end of the housing 130 by turning the cover 150 by 180 degrees to the other side of the coupling portion 160 (\textcircled{4}).

The user brings the lancet device 100 ready for taking blood in contact with a portion to take blood from, such as a finger, in which the protrusions 153 press the area around the portion, such that nerves around are stimulated.

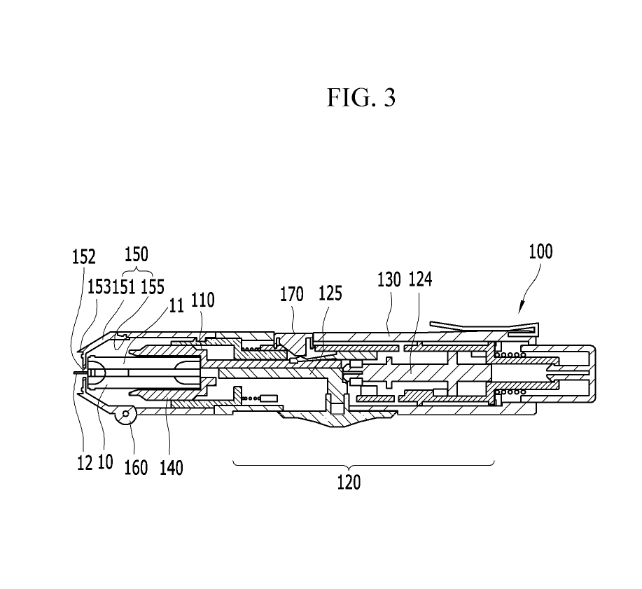

Finally, when the user operates the operating mechanism 120 by pressing the operation switch 170 (see FIG. 3), the lancet 10 is released and the lancet needle 12 penetrates the skin, thereby taking blood.

The process of opening the cover 150 to remove the lancet 10 from the lancet device 100 after taking blood is also performed in the same way.

The above description is an example of the spirit of the present invention and may be changed and modified in various ways by those skilled in the art without departing from the scope of the present invention. Therefore, the exemplary embodiments described herein are not for limiting the spirit of the present invention, but for explaining the present invention and the scope of the present invention is not limited to the exemplary embodiments. The protective range of the present invention should be construed by claims and the equivalents should be construed as being included in the scope of the present invention.

While this invention has been described in connection with what is presently considered to be practical exemplary embodiments, it is to be understood that the invention is not limited to the disclosed embodiments, but, on the contrary, is intended to cover various modifications and equivalent arrangements included within the spirit and scope of the appended claims.

What is claimed is:

1. A lancet device that includes:
a lancet holder having a lancet seat to mount a lancet at one end;
an operating mechanism disposed at the other end of the lancet holder and loading and releasing the lancet holder along a predetermined path; and
an a housing receiving the operating mechanism and having one end coupled to the lancet holder,
the device comprising a cover that covers the housing so that the lancet holder is received in the housing, is at least partially connected with the housing in order not to separate from the housing in use, and has protrusions on the front part.

2. The device of claim 1, wherein the cover has a through-hole at the center through which a lancet needle of the lancet instantaneously protrudes, and has a coupling portion that hinges the cover to one end of the housing.

3. The device of claim 1, wherein the cover has a locking protrusion that is formed at the other end of the front part and fitted in a fitting groove formed at one end of the housing.

4. The device of claim 1, wherein the protrusions are radially formed at regular angles.

5. The device of claim 4, wherein the protrusions are alternately arranged on a plurality coaxial circles.

6. The device of claim 4, wherein the protrusions have a triangular shape.

7. The device of claim 5, wherein the protrusions are alternately arranged in two lines.
